(19) United States
(12) Patent Application Publication
(10) Pub. No.: US 2005/0102009 A1
(43) Pub. Date: May 12, 2005

(54) Ultrasound treatment and imaging system

(76) Inventor: Peter Costantino, New York, NY (US)

Correspondence Address: ANTHONY H. HANDAL;KIRKPATRICK & LOCKHART NICHOLSON GRAHAM LLP, 599 LEXINGTON AVENUE, 33RD FLOOR, NEW YORK, NY, 10022-6030, US

(21) Appl. No.: 10/903,323

(22) Filed: Jul. 30, 2004

Related U.S. Application Data
(60) Provisional application No. 60/491,601, filed on Jul. 31, 2003.

(51) Int. Cl. A61F 7/00; A61F 7/12; A61F 2/00; A61B 5/05

(52) U.S. Cl. 607/96; 600/411

(57) ABSTRACT

Apparatus for treating cellulite under the surface of the skin comprising a surgical ultrasonic energy source is disclosed. An ultrasonic energy transmitting surface is driven by a surgical ultrasonic energy source. The ultrasonic energy transmitting surface is configured to concentrate ultrasonic energy in the layer of connective tissue lying at the interface between the dermis and the subcutaneous adipose tissue. The surgical ultrasonic energy source is of sufficient power to cause disruption of the layer of connective tissue lying at the interface between the dermis and the subcutaneous adipose tissue during a dosage period. An imaging ultrasonic energy transmitting surface is driven by the imaging ultrasonic energy source. An imaging ultrasonic energy detector is coupled to receive the reflected output of the imaging ultrasonic energy transmitting surface. A computing device is coupled to the output of the imaging ultrasonic energy detector. A program resident in the computing device configures the computing device to generate an image of tissues under the surface of the skin of a patient. A display for displaying the image of tissues under the surface of the skin of the patient provides the person implementing the treatment with first and second images. The first image is a before ultrasound treatment three dimensional or other image and the second image is an after ultrasound treatment image of the tissue is treated. An applicator housing is configured to be held by the hand of an individual, the imaging ultrasonic energy detector is mounted on the applicator housing.

### CROSS REFERENCE TO RELATED APPLICATIONS

This application claims the benefit of U.S. provisional patent application No. 60/491,601, filed Jul. 31, 2003, (Attorney Docket No. PDC019) the entire disclosure of which is hereby incorporated herein by reference thereto.

### STATEMENT REGARDING FEDERALLY SPONSORED RESEARCH OR DEVELOPMENT

Not Applicable.

### BACKGROUND OF THE INVENTION

1. Field of the Invention

The invention relates both to 1) an ultrasonic apparatus for treating patients suffering from cellulite, (a condition characterized by portions of subdermal adipose fat-containing tissue migrating into and through the subdermal elastic tissue matrix) and for the purpose of causing the contraction of laxed or wrinkled tissues below the surface of the epidermis, and also to 2) a method using ultrasonic energy to non-invasively generate a relatively robust connective tissue matrix at the interface of the dermis and the adipose tissue.

2. Prior Art

The distribution of adipose tissue throughout the body is not uniform. In certain portions of the body, such as in the subcutaneous tissue, it is present in great abundance. A distinction may be made between fat and adipose tissue, adipose tissue being a distinct type of tissue, and fat merely being an oily substance, found, for example in a fat cell.

Adipose tissue consists of small vesicles or “fat cells” lodged within a matrix of areolar connective tissue. Fat cells vary greatly in size; having an approximate diameter of about 0.05 mm. They are formed of a delicate protoplasmic membrane filled with an oily liquid substance. These fat cells are contained in discrete clusters in the areolae of fine connective tissue.

Areolar tissue is a form of connective tissue in which the investing connective tissue matrix is separated into areolae or spaces which open into one another and are easily permeated by fluids. Areolar tissue binds different parts of the body together. The elasticity of areolar tissue and the permeability of its areolae allows the various parts of the body to move relative to one another.

Areolar connective tissue is found beneath the skin in a continuous layer all over the body, connecting the skin (dermis) to subjacent tissues. In many parts of the body, the areolae are occupied by fat cells. The matrix and fat cells constituting adipose tissue are referred to alternatively herein as “depot fat”. It is now well established that the phenomena of cellulite is due to a protrusion of pockets of depot fat, from subcutaneous tissue through an elastic layer of connective tissue overlying the depot fat, into the dermis. Patients having cellulite appear to exhibit a deficiency in the fibrous layer at the interface between the dermis and the subcutaneous tissue.

This deficiency allows depot fat below the interface to protrude up through the fibrous layer and into the dermis, thereby causing irregular elevations and depressions of the dermis characterized by a “dimpled” appearance. This condition, and the underlying causes, is discussed by Rosenbaum et al. in Plastic and Reconstructive Surgery, Vol. 104. No. 7, Pages 1934-1939, June, 1998.

Ultrasonic, microwave, nuclear magnetic resonance and other radiative techniques have been employed to provide images of organs within the body and/or to effect treatment of subdermal tissue without necessitating traumatic incision of the overlying tissue. Radiative application may have as its object the destruction of target cells in a particular target area beneath the skin or the imaging of organs, tumors or other structures for diagnostic purposes.

Therapeutic applications include the cauterization of blood vessels via hyperthermia for traumatic injury resulting in bleeding, as occurs, for example, in trauma to the liver. In addition, such radiative treatment may be employed for diagnosing medical conditions, treating prostate hypertrophy, non-invasive lipectomy or for the treatment of brain cancer.

Various devices have been designed and disclosed for performing these and other procedures. Exemplary of such devices is U.S. Pat. No. 5,769,790 to Watkins, et al.; U.S. Pat. No. 5,143,063 to Fellner and U.S. Pat. No. 5,507,790 to Weis.

Knowlton, in U.S. Pat. No. 5,755,753, discloses a method for tightening skin. The method comprises providing a membrane containing a cooling fluid in combination with a thermal energy source. A reverse thermal gradient is created which cools the surface of the skin while heating underlying collagen-containing layers of tissue. The skin and underlying collagen-containing tissue are then heated without substantially modifying the melanocytes and other epithelial cells in the epidermis. The result is a contraction of collagen tissue and a tightening of the skin. Radiant energy is applied to a variety of different skin layers including the papillary dermis layer, the reticular dermis layer, and even to a subcutaneous layer and to underlying soft tissue. A suitable energy source is one or more radio frequency electrodes. The electrolytic solution contained within the membrane transfers radio frequency energy from the radio frequency electrodes to the underlying collagen tissue. The cooling fluid creates a reverse thermal gradient between the epidermis and the underlying desired layers of about thirty to about eighty degrees centigrade. The creation of the reverse thermal gradient provides for the controlled contraction of collagen tissue, e.g., partial denaturization of the collagen molecules that results in a shrinkage of the collagen tissue, which then extends to a shrinkage of the skin.

Creation of the reverse thermal gradient is different from other methods of collagen contraction which typically employ a thermal gradient that has a higher temperature at the surface and decreases with the depth of penetration. Thus, Knowlton's device and method for causing shrinkage of the skin requires cooling the epidermis while heating collagen in the underlying tissue via radiant means such as a radiofrequency field.

Knowlton '753 addresses the problem of tightening the skin by increasing cross-linking in collagen in a selected target layer of tissue beneath the skin. That is, the method of Knowlton does not stimulate production of collagen by cells within the target tissue. The method, instead, relies upon increasing the cross linking between amino acids in adjacent collagen fibrils. While Knowlton '753 does not present data specifying a temperature threshold above which the objectives of the method are achieved, the thermoregulating ability of the body renders it difficult, if not impossible, to raise the temperature of a layer of tissue underlying the cooled epidermis to a point where denaturation of collagen will occur by employing non-invasive hyperthermia means.

The present invention provides a method for stimulating the production of additional collagen in a preselected target area thereby increasing the collagen content in the tissue.

### SUMMARY OF THE INVENTION

It is a first object of this invention to provide an apparatus creating controlled tissue injury and repair for structurally reinforcing one or more layers of connective tissue beneath the skin of a patient. It is another object of this invention to create or reinforce a layer of connective tissue at the interface between the dermis and the subcutaneous tissue of a patient by non-invasive means. It is yet another object of this invention to provide a method for enhancing the integrity of a connective tissue layer beneath the dermis to prevent lobules comprising adipose tissue from protruding into the layer of skin comprising the dermis. It is a further object of this invention to provide a means for strengthening the fibrous layer of tissue at the interface between the dermis and the subcutaneous tissue to reduce or prevent cellulite. The features of the invention believed to be novel are set forth with particularity in the appended claims.

In accordance with the invention, apparatus for treating cellulite under the surface of the skin of a patient comprises a treatment ultrasonic energy source. An ultrasonic energy transmitting surface is driven by the treatment ultrasonic energy source. The ultrasonic energy transmitting surface is configured to concentrate ultrasonic energy in the layer of connective tissue lying at the interface between the dermis and the subcutaneous adipose tissue. The treatment ultrasonic energy source is of sufficient power to cause disruption of the layer of connective tissue lying at the interface between the dermis and the subcutaneous adipose tissue during a dosage period.

An imaging ultrasonic energy transmitting surface is driven by the imaging ultrasonic energy source. An imaging ultrasonic energy detector is coupled to receive the reflected output of the imaging ultrasonic energy transmitting surface. A computing device is coupled to the output of the imaging ultrasonic energy detector. A program resident in the computing device configures the computing device to generate an image of tissues under the surface of the skin of a patient. A display for displaying the image of tissues under the surface of the skin of the patient provides the person implementing the treatment with first and second images. The first image is a before ultrasound treatment three dimensional or other image and the second image is an after ultrasound treatment image of the tissue is treated. An applicator housing is configured to be held by the hand of an individual, the imaging ultrasonic energy detector is mounted on the applicator housing. The imaging ultrasonic energy transmitting surface is mounted on the applicator housing, and the treatment ultrasonic energy transmitting surface is mounted on the applicator housing.

In accordance with the preferred embodiment, the concentration of electronic energy during the dosage period results in a dosage sufficient to result in the necrosis of cells in the connective tissue and cause the introduction of fibroblasts and the formation of new connective tissue sufficient to reinforce the layer of connective tissue to a degree where the strength of the connective tissue exceeds the strength of the connective tissue prior to the application of the dosage of ultrasound.

The treatment ultrasonic energy transmitting surface is driven by a piezoelectric device. Preferably, the ultrasonic energy transmitting surface is configured to be in contact with the surface of the skin of the patient. The treatment ultrasonic energy transmitting surface may focus energy in a linear area.

The display is preferably mounted on the applicator housing. The imaging ultrasonic energy source and treatment ultrasonic energy source are alternately actuated in an embodiment of the invention. The program may determine, plan and control the administering of ultrasound. The applicator housing comprises indicators that guide directional movement of the applicator housing by the person implementing the treatment.

An accelerometer may be coupled to the computing device to input information respecting applicator housing movement during the application of treatment ultrasound to the computing device, and software associated with the computing device generates a signal coupled to the display indicating the region of application of treatment ultrasound to tissue to be treated.

The treatment ultrasonic energy transmitting surface may alternately comprise a line of discrete energy transmitting members. A logic device controls the treatment ultrasonic energy source to drive a selectable one of the discrete energy transmitting members in response to determined dosage amounts. The apparatus may be caused to scan back and forth across the line of discrete energy transmitting members and successively alter cellular structure at the interface portion of the fat depot and the epidermis, whereby the person implementing the treatment may evaluate the progress of the treatment as it proceeds.

Preferably, the rate at which ultrasound is performed is commensurate with the rate at which the person implementing the treatment is able to monitor and evaluate the effects of ultrasound treatment.

The display may, optionally provide the person implementing the treatment with first and second images. The first image is a before ultrasound treatment three dimensional or other image. The second image is an after ultrasound treatment image of the tissue.

A pressure applying member is configured to apply pressure to the skin area overlying the surface of the skin of the patient is treated and comprises a rigid member bearing against the skin of the patient and a pressure member urging the rigid member against the skin of the patient.

In an alternate preferred embodiment, the concentration of ultrasonic energy focuses that energy in selectable portions of a linear area.

### BRIEF DESCRIPTION OF THE DRAWINGS

The invention itself, both as to organization and method of operation together with further objects and advantages thereof may best be understood by reference to the following description taken in conjunction with the accompanying drawings, in which:

FIG. 1 is a schematic diagram of the inventive ultrasound administering and imaging system;

FIG. 2 is a perspective view of a hand-held imaging and ultrasound treatment administering device useful in the system illustrated in FIG. 1;

FIG. 3 is a top plan view of the administering device of FIG. 2 showing the controls and the display;

FIG. 4 is a bottom plan view of the administering device of FIG. 2 illustrating the transducers and detectors;

FIG. 5 is a flowchart illustrating the operation of the system of FIG. 1;

FIG. 6 is a perspective view of a rigid pressure applying member;

FIG. 7 is a plan view of an elastic member for providing pressure to the rigid pressure applying member of FIG. 6;

FIG. 8 is a view, along the lines 8-8 of the elastic member of FIG. 7;

FIG. 9 is a schematic cross-sectional view depicting the human epidermis, dermis and subcutaneous tissue;

FIG. 10 is a perspective view of a skin-contacting portion of an ultrasonic vibrator scanner hand-piece suitable for the application of ultrasonic energy to tissues at the interface between the dermis and subcutaneous tissue;

FIG. 11 is an end-on view of the skin-contacting portion of the ultrasonic vibrator hand-piece of FIG. 10 together with a schematic block diagram of an apparatus for applying radiant or ultrasonic energy to create or reinforce a layer of connective tissue in accordance with the principals of the present invention;

FIG. 12 is a block diagram of an apparatus suitable for applying ultrasonic energy to the interface between the dermis and the subcutaneous tissue of a patient;

FIG. 13 is a front elevational view of a preferred embodiment of the ultrasonic vibrator hand-piece of the present invention; and

FIG. 14 is a side elevational view of a preferred embodiment of the ultrasonic vibrator hand-piece of the present invention.

### DETAILED DESCRIPTION OF THE PREFERRED EMBODIMENTS

In accordance with the present invention, the elastic layer of connective areolar tissue overlying the depot fat, at the interface of the adipose cell layer and the dermis, is damaged to stimulate the introduction of fibroblasts and the formation of a more dense layer of connective tissue which defines smaller interstices and thus is more effective to maintain the depot fat below the interface, as compared to the condition in patients suffering from cellulite, where the depot fat is protruding out from the elastic layer and accumulate directly under the dermis, forming globules of adipose tissue which are visible as small lumps under the skin.

The controlled and focused application of traumatic ultrasonic waves to cells comprising a thin layer of tissue adjacent to and including the interface between the dermis and subcutaneous adipose tissue results in tissue destruction followed by an inflammatory response accompanied by the migration of fibroblasts into the area. The similar use of such controlled and focused traumatic energy could be used to create a like tissue response fully within the subcutaneous fat or dermis depending upon the “individualized clinical” condition of the patient under treatment. The intentional directed infiltration of fibroblasts to the thin layer of tissue including the interface may be used to reinforce the structural integrity thereof resulting in an amelioration or elimination of subdermal fat protrusion (cellulite). Several techniques are effective to increase the reduction of cellulite in accordance with the invention. In particular, in accordance with the invention, cellulite is reduced not by the destruction of the fat cells which form the cellulite, but by relocation of the fat cells into the adipose tissue on the other side of the elastic interface layer, where the bulk of the depot fat is located, even in the case of most individuals suffering from cellulite.

In accordance with the inventive method, the inventive apparatus concentrates treatment ultrasonic energy in the layer of connective tissue lying between the dermis and the subcutaneous adipose tissue to perform a surgical function, namely, tissue damage, while at the same time imaging ultrasound is used to produce an image of the area being operated on by the physician. Treatment ultrasound can be adapted to not interfere with imaging ultrasound by time-multiplexing the two ultrasound emissions, that is to say treatment ultrasound can be produced after a burst of imaging ultrasound, and imaging ultrasound can be produced after a burst of treatment ultrasound, with the two ultrasound signals alternating without overlapping in time. The inventive treatment ultrasonic energy source is of sufficient power to cause disruption of the layer of connective tissue during application of a clinical dosage of ultrasound. In accordance with the invention, the dosage may be sufficient to result in the necrosis of cells in the connective tissue. This results in the introduction of fibroblasts by the normal healing mechanisms of the body. The introduction of fibroblasts is followed by the formation of new connective tissue sufficient to reinforce the layer of connective tissue to a degree where the strength of the connective tissue after the body's repair process exceeds the strength of the connective tissue prior to the application of the dosage of ultrasound.

In accordance with the invention, the inventive process may begin with the application of pressure to the skin in the area to be treated with ultrasound, prior to the application of ultrasound. The application of pressure may be made by applying a hard curved object which roughly conforms to the shape of the body portion suffering from cellulite. Such a roughly conforming rigid member may be placed against the skin in the affected area and elastic bandaging used to compress the rigid member against the area to be treated. After a number of hours or days or longer, depending upon the time which the cellulite has been allowed to continue, the characteristics of the connective layer of tissue in the individual patient, and the amount of cellulite to be relocated, cellulite under the influence of pressure tends to migrate from the region between the connective areolar tissue and the dermis. Under pressure fat cells forming cellulite are caused to migrate through the connective layer of tissue into the layer of depot fat.

The advantage of applying pressure prior to the application of ultrasound is that pressure may be applied for an extended period of time. When the elastic bandage is removed, the fat cells, which formerly formed the cellulite, tend to remain in their proper position in the depot fat. Alternatively, or in addition, pressure may be applied in the same or other ways (such as simple elastic bandages, textile sheets formed into constrictive pressure applying tubes through the use of a Velcro brand hook and loop closure, or the like) after the application of ultrasound.

In the case of pressure applied after the application of treatment ultrasound, the migration of cellulite forming fat cells into the depot fat may be improved due to the damage done to the connective layer of tissue.

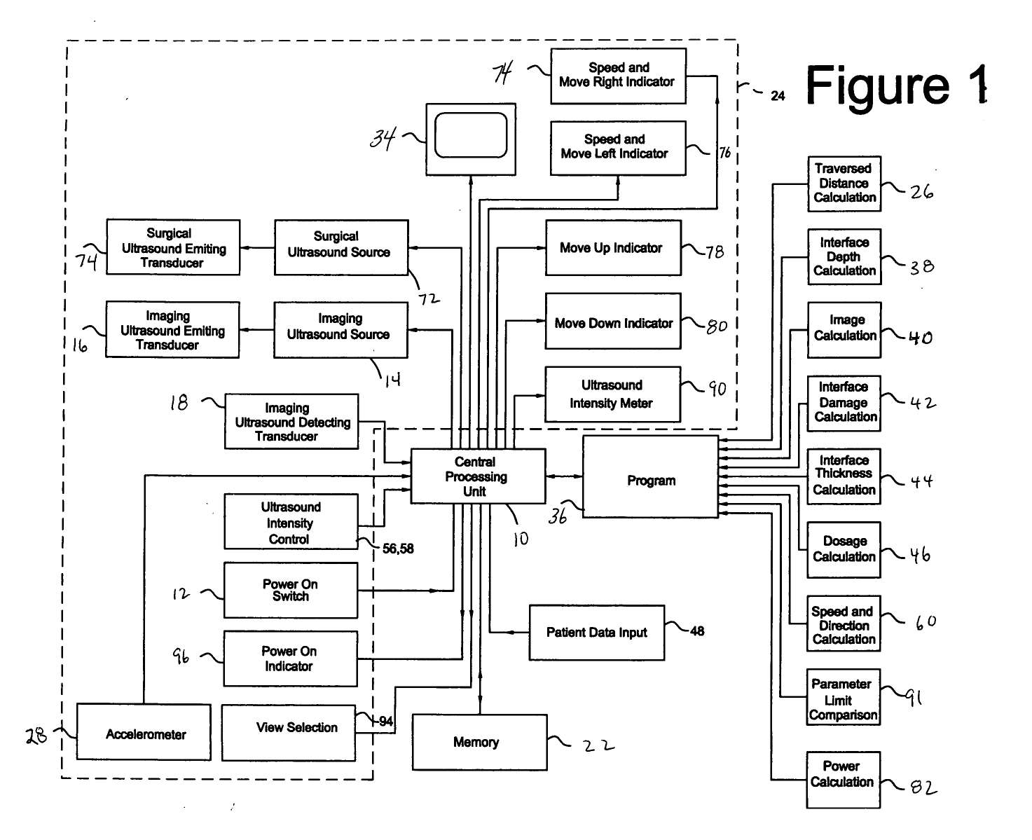

The inventive system is illustrated in FIG. 1. The system is controlled by a central processing unit 10 which may be a dedicated microprocessor, or, more preferably, a general-purpose computer of the type incorporating memory in the form of a hard drive and random access memory, and incorporating interface circuitry such as a USB port, card slot, motherboard printed circuit card slot or the like, as would be provided by a personal computer or, if portability is desired, a laptop computer, for example one centered on a Pentium IV microprocessor.

Central processing unit 10 is responsive to a switch 12 to turn on the system, or reset the system, if it has ceased producing treatment ultrasound, as is described more fully below. Switch 12 is coupled to central processing unit 10 by any interface such as a USB port. Central processing unit 10 also drives imaging ultrasound source 14 which produces an electrical signal which drives imaging ultrasound emitting transducer 16.

Imaging ultrasound produced by ultrasound emitting transducer 16 is coupled to the skin overlying the cellulite to be treated. Reflected ultrasound echoes are detected by imaging ultrasound detector 18 which couples them through the interface to central processing unit 10. Information gathered by transducer 18 is processed by central processing unit 10 which, in turn, sends information into memory 22 for storage.

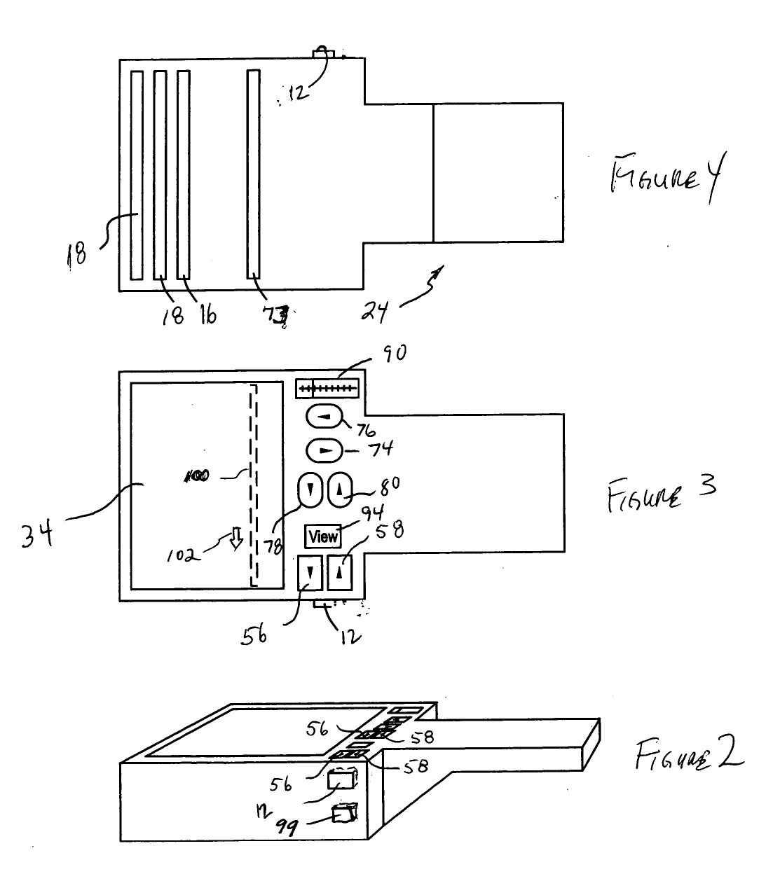

As will be described below in connection with FIGS. 2 through 4, pattern recognition is used to determine the distance traversed by a portable skin imaging and treating device 24. Imaging and treatment ultrasound applying handpiece 24 may be wire coupled to central processing unit 10, or coupling may be by wireless or infrared connection. This may be done using a calculation subroutine 26 or the output of an accelerometer 28 mounted on imaging and treatment ultrasound applying hand-piece 24. Traverse distance calculation subroutine 26 is one of a number of modules in an overall program 36 which central processing unit 10 uses to control the inventive system illustrated in FIG. 1.

During use, imaging and treatment ultrasound applying hand-piece 24 is positioned with transducer 18 in contact with the cellulite exhibiting skin of the patient and scanned over an area to be imaged and treated. By calculating traversed distance, a coherent picture of the physiology may be produced and displayed on a display, such as liquid crystal display 34, which is mounted on imaging and treatment ultrasound applying hand-piece 24. Alternatively, or in addition, the display associated with the laptop or other computer may be used to display the underlying tissue. Use of both a display on imaging and treatment ultrasound applying hand-piece 24 and the standard display on the computer may be of value for the greater degree of detail which may be provided on the standard computer display, which may, for example, be a 17 inch flat-panel display, as compared to the relatively small display on imaging and treatment ultrasound applying hand-piece 24. However, the display 34 carried on board imaging and treatment ultrasound applying hand-piece 24 has the value of being visible while the doctor is performing the procedure.

Both the images on display 34 and on the laptop or other personal computer screen may be color-coded to show the cellulite, and may be a simple cross-sectional display, or a three-dimensional perspective or orthogonal view.

Program 36 connects with other modules including a subroutine 38 for calculating interface depth, and a subroutine 40 for calculating an image in three dimensions. Changes in the image of the cellulite overtime may be used to calculate the amount of damage done to a particular portion of the subdermal region being treated, and display the same in the image. Such calculation is done by image interface damage calculation subroutine 42.

The inventive system also provides for calculation of the interface thickness, that is the thickness of the interface layer which is to be damaged. This information, generated by subroutine 44, allows the focusing of the ultrasound over the proper range of thickness and together with the output of subroutine 38 controls the intensity and region of application of the ultrasound output.

The magnitude of the ultrasound output is calculated using dosage calculation subroutine 46. Calculation of dosage at subroutine 46 is subjected to testing against certain norms stored in memory 22. These norms are a function of patient input data which is input through the use of alphanumeric keyboard 48 coupled to the central processing unit 10 in conventional form, through the use, for example, of the keyboard and alphanumeric prompts on display 34 or the display on the computer. Such input patient data optionally includes age, severity of cellulite, sex and so forth. If desired the physician may use manual controls 56 and 58 to either increase or decrease in the level of ultrasound application manually. However, it is contemplated that the level of ultrasound may be controlled by central processing unit 10 which will also direct the user with respect to the speed and direction of the application of ultrasound through the use of a speed and direction calculation subroutine 60, which drives a number of indicators.

In accordance with the invention, it is contemplated that a treatment ultrasound source 72 will be actuated by central processing unit 10 to deliver ultrasound to achieve the desired amount of cell damage. This ultrasound signal is used to drive treatment ultrasound emitting transducer 73, which produces the ultrasound which is coupled to the skin in the cellulite affected area.

These indicators include indicator 74 which indicates to the physician that he should move to the right. Blinking of the indicator may be used to indicate that greater scanning speed is required in the movement of imaging and treatment ultrasound applying hand-piece 24. Likewise, indicator 76 indicates movement to the left with blinking indicating the need for greater scanning speed. Slower scanning speed is not a problem because the system can simply disable treatment ultrasound source 72, thus reducing the output of treatment ultrasound emitting transducer 73 to zero until movement indicates that the application of more ultrasound is required.

In similar fashion, the system provides a move up indication by illuminating a move up indicator 78. If downward movement is required, move down indicator 80 indicates a downward direction with the orientation of its arrow and when illuminated indicates to the physician that he should move down. It is noted that all arrows in the indicators correspond to the desired motion of the imaging and treatment ultrasound applying hand-piece.

Subroutine 82 calculates the amount of power to be delivered by treatment ultrasound transducer 73. However, even after the system has determined that the desired amount of ultrasound has been applied, the physician still has the option of operating manual controls 56 and 58 to achieve a desired level of treatment ultrasound application. In this case, the display of ultrasound intensity provided by meter 90 is of particular importance. If desired, the physician may select between different views using switch 94, by repeatedly pressing of switch 94 to sequence through different displays for presentation on display 34. As will be described below, the system will shut itself down in response to the detection of a potentially dangerous condition. The physician is aware of whether the system is functioning or not through the actuation of a power-on indicator light 96, which is extinguished when the system shuts down.

As alluded to above, the inventive system essentially comprises an imaging and treatment ultrasound applying hand-piece 24, as illustrated in FIGS. 24. Imaging and treatment ultrasound applying hand-piece 24 carries transducers 16, 18 and 43, sources 14 and 72, display 34, a view selector switch 94, indicators 74, 76, 78,80 and 96, an ultrasound intensity meter 90, controls 56 and 58, an accelerometer 28, and on switch 12.

Turning on of imaging ultrasound and disabling of the treatment ultrasound is accomplished by the pressing of a button 99. This allows imaging and treatment ultrasound applying hand-piece 24 to be used to generate only an image of the area to be treated preparatory to the use of treatment ultrasound to damage the interface below the epidermis. The exact area which is being treated by treatment ultrasound may be displayed to the physician by being highlighted or contained in an area indicator 100.

Treatment ultrasound emitting transducer 73 may, optionally, be comprised of a line of discrete members, for example, five pixels with a dimension of 1 cm by 1 cm. The system may, optionally, actuate the first pixel at one end that is to emit ultrasound and then move on to the next, with an arrow indicator 102 being displayed on the screen indicating to the physician that damage is proceeding in one direction or another.

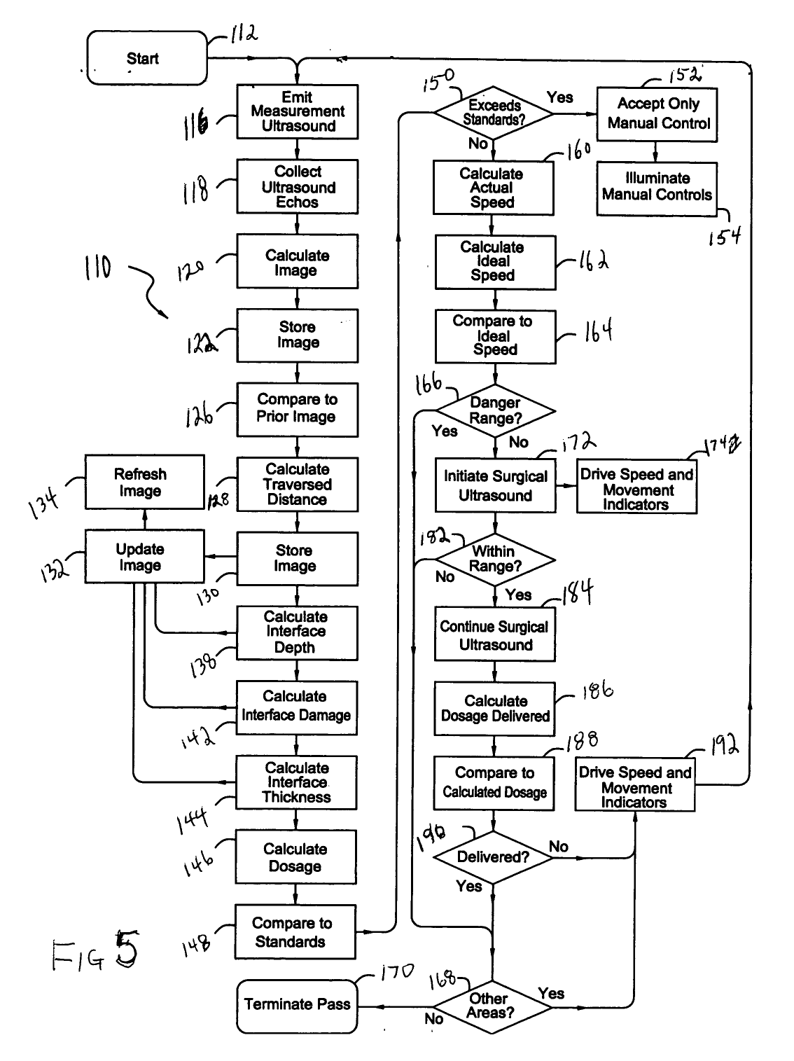

The method 110 of the present invention may be understood with reference to FIG. 5. Where practical, method steps have been numbered with numbers 100 higher than the numbers of related apparatus elements in FIGS. 1-4, for ease of reference and understanding.

Method 110 is controlled by central processing unit 10. System operations are initiated at start step 112, with the actuation of switch 12, causing the system at step 116 to actuate imaging ultrasound source 14 causing imaging ultrasound emitting transducer 16 to emit an ultrasound signal which is conducted through the tissues to generate echoes which are detected at step 118 by imaging ultrasound detecting transducer or transducers 18. The use of multiple transducers results in generating redundancy and diversity in the information, thus allowing the generation of a relatively high-resolution and more accurate image.

Initially, the physician scans an area which is to be treated. After the system determines that area has been scanned and the physician is beginning to retrace over the same area for the purpose of applying ultrasound, the system proceeds as is described below to determine dosage and apply the same, or default to manual control.

At step 120 collected ultrasound echoes are processed using conventional techniques to calculate an image of the tissues using conventional techniques. The image is stored at step 122 in memory 22. The present invention relies on the detection of speed of movement of manual imaging and treatment ultrasound applying hand-piece 24. Accordingly, at step 126 the most recent previously stored image is compared to the image stored at step 122. Comparison of images allows calculation of the distance traversed by imaging and treatment ultrasound applying hand-piece 24 at step 128. This information may be cross-checked with information output by accelerometer 28, if an accelerometer is included in the system. Of particular value in the case of administering large doses of ultrasound, in addition to redundancy, accelerometer 28 offers the feature of high-speed detection of movement.

Comparison of images at step 126 may result in additional information respecting the image, and this additional information may, optionally, be included in an improved image which is stored at step 130. Also at step 130, the most recent image is used to update the screen display at step 132, resulting in driving the display 34 at step 134 with the most recent image. Speed may be calculated using program 36, subroutine 26.

In accordance with the present invention, it may be desired to graphically illustrate the elastic layer of connective areolar tissue overlying the depot fat, at the interface of the adipose cell layer and the derma, in order to gauge its location and the amount of damage done to the layer of connective tissue. This may be done using program 36, subroutine 38 at step 138. In accordance with the preferred embodiment, at step 138, subroutine 40 is used to calculate an image of the layer of connective tissue. Cellular interface damage is calculated at step 142 using subroutine 42. At step 144, interface thickness is calculated using subroutine 44. Image information respecting interface steps and interface damage calculated at step 138 and 142 may then be sent at step 132 to the system to refresh the image at step 134.

The system then proceeds at step 146 to calculate the dosage of ultrasound needed to cause the desired amount of damage using subroutine 46. The dosage calculated at step 146 is then compared at step 148 to a set of standards stored in memory 22. Such standards are based upon the height, weight and age of the patient, input into the system by information input interface 48, which may be a keyboard, display screen in the program for the input of information by clicking, or combinations of the same, or the like. The purpose of the standards is to provide rough but safe guidelines to prevent the administration of dangerous overdosages of ultrasound. The standards stored in memory relate to the amount of connective interface tissue likely to be found, its depth, and its thickness. These act as a rough check on computer calculated information and prevents computer error or measurement errors caused by, for example, failure to bring imaging and treatment ultrasound applying hand-piece 24 into contact with the skin of the patient, or other abnormal condition or conditions.

In accordance with the present invention, if at step 150, the dosage calculated at step 146 exceeds the standards, the system proceeds to step 152 where the automatic actuation of ultrasound administration is disabled. At step 154, manual controls 56 and 58 are illuminated.

Based upon the image comparison made at step 126 or accelerometer data collected from accelerometer 28, the actual speed of imaging and treatment ultrasound applying hand-piece 24 is calculated at step 160. Based upon physiologic data on the layer of connective tissue and damage done or to be done to the layer, the system calculates an ideal speed for movement of imaging and treatment ultrasound applying hand-piece 24 at step 162. At step 164 this ideal speed is compared to the actual speed calculated at step 160 using subroutine 60.

If the difference indicates that, given the amount of damage already done to the layer of connective tissue, any application of ultrasound may be dangerous, the system, at step 166, terminates the application of treatment ultrasound in a given area and consults memory 22 to determine whether any other areas which require ultrasound treatment at step 168. If there are no such areas, the pass is terminated at step 170. Information on areas which require ultrasound may be generated by the system, or maybe input by the physician by using a stylus on-screen 34. Likewise, screen 34, in accordance with the present invention indicates the area where destructive treatment ultrasound is being applied.

If, on the other hand, the difference indicates that, given the amount of damage already done to the layer of connective tissue, application of ultrasound by the automatic system is appropriate, at step 166 the system proceeds to step 172, where treatment ultrasound source 72 drives treatment ultrasound emitting transducer 73 with a level of treatment ultrasound calculated at step 82. In order to prevent confusion between the surgical and the imaging ultrasound sources, surgical and imaging ultrasound are applied at different frequencies or at different times, thus allowing the selective evaluation of reflected imaging ultrasound.

Simultaneous with the initiation of treatment ultrasound at step 172, speed and movement indicators 74-80 are driven at step 174. If the further application of ultrasound is within the ranges indicated by patient data input using interface 48, the system proceeds at step 182 to go to step 184 where treatment ultrasound is continued.

At step 186, the delivered dosage is again calculated and compared at step 188 to the desired dosage, calculated at step 146, which one wishes to deliver. At step 191, if the dosage is not been delivered, the system proceeds using subroutine 91 to drive speed and movement indicators 74-80 at step 192. If the dosage has been delivered, the system checks at step 168 to determine if there are other areas. If there are other areas the system proceeds to step 192. After step 192, the system proceeds to step 116 and repeats the process as described above.

Even after the system has determined that the desired amount of ultrasound has been applied, the physician still has the option of operating manual controls 56 and 58 to achieve the desired effect. In this case, the display of ultrasound intensity provided by meter 90 is of particular importance. If desired, the physician may select between different views using switch 94, repeated pressing of which will sequence different displays for presentation on display 34. In accordance with the present invention, the surgeon who wishes to use the inventive apparatus presses button 99 on imaging and treatment ultrasound applying hand-piece 24. The pressing of button 99 disables the treatment ultrasound and enables only the imaging ultrasound, thus providing for scanning the area to be treated. The result is to generate within the memory 22 of central processing unit 10 a picture of the volume of tissue being treated.

The physician may then use a stylus on-screen 34 to indicate those areas where cellulite is to be reduced. Alternatively, the system may analyze the image and provide recommendations to the physician.

Next the surgeon releases button 99 and presses button 12 to initiate the application of ultrasound by transducer 73. Application of ultrasound is automatic, but may be overridden from a zero or any value condition using buttons 56 and 58. The surgeon may also be provided with an area indication 100 on display 34, which indicates where ultrasound is being applied.

In accordance with the preferred embodiment, it is also contemplated that treatment ultrasound emission transducer 73 may be made of a plurality of separate discrete members, only some of which may be activated at a given time, in order to provide the desired degree of ultrasound in various parts of the area being treated.

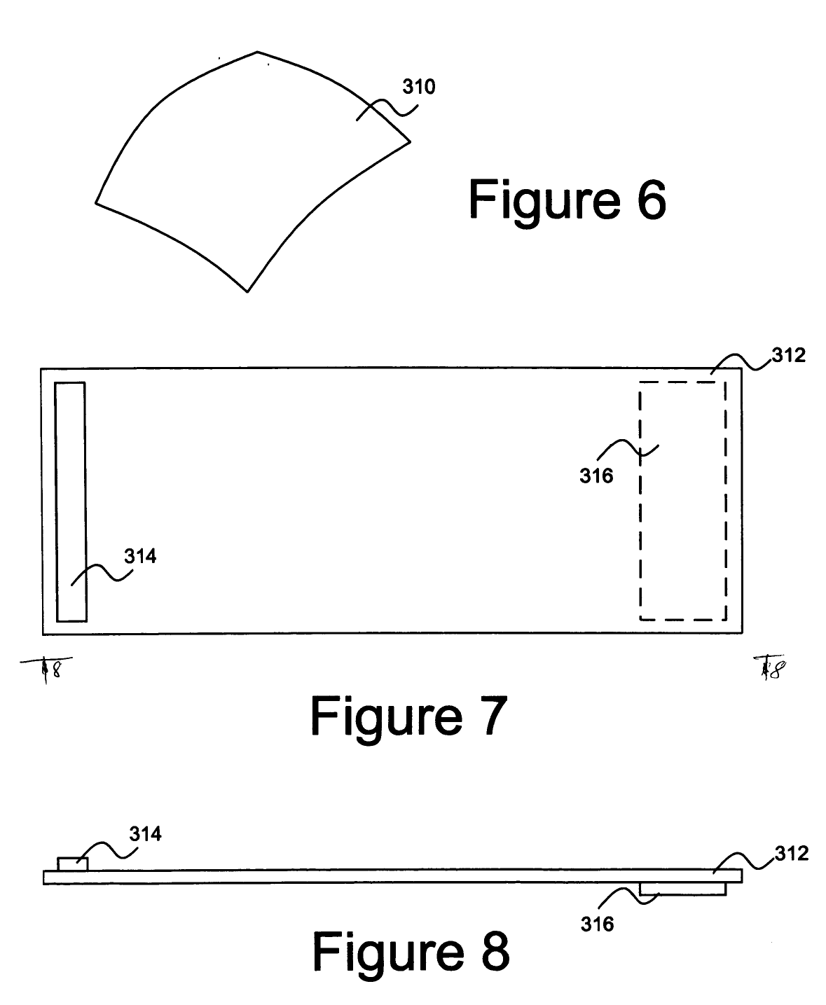

In accordance with the invention pressure may be applied either before or after the application of treatment ultrasound. Pressure may be applied using a rigid plate 310, as illustrated in FIG. 6. Plate 310 is roughly formed to conform to the shape of the body. Pressure is applied to plate 310 by any suitable constricting member, such as band 312, which may be made of an elastic material and which may be wrapped around a body part and secured to itself to form a constrictive elastic tube by causing Velcro hook mating member 314 to come into contact with loop mating member 316.

In accordance with a preferred embodiment of the invention, the damage produced by treatment ultrasound emitting transducer 73, which may be comprised of a number of discrete members, may be limited to a single pixel of dimension in the range between 0.25 cm on a side and 5 cm on a side (for example 0.25 cm×0.25 cm, or 0.25 cm×0.5 cm), but preferably in the range between 0.5 cm on a side and 2 cm on a side, but preferably in the range between 0.5 cm on a side and 2 cm on a side, and most preferably in the range between 0.75 cm on a side and 1.5 cm on a side.

In accordance with this optional feature, a physician can concentrate on a small area to monitor the progress of the ultrasound treatment. More particularly, it is contemplated that the system will scan back and forth across the line of treatment ultrasound emitting transducers and successively alter cellular structure at the interface portion of the fat depot and the epidermis. This allows the physician to evaluate the progress of the treatment as it proceeds. Accordingly the rate at which ultrasound is performed should be commensurate with the rate at which the physician is able to monitor and evaluate progress.

Optionally, the physician may be provided with two images on display 34, namely a before ultrasound treatment three dimensional or other display and an after ultrasound treatment display of the tissue being treated. The before treatment display may be the images obtained at the beginning of the session, or it may be stored images from any point in time, such as a few months before the current treatment and perhaps before treatments given after the earlier image but before the present. The extent of damage to the cells can be determined from differences in successive images over time (which changes are likely to be substantially solely due to the ultrasonic treatment).

At a point in the treatment, the system will indicate to the physician that the area has been treated and the physician is directed to move to the right or left. Conversely, the physician may override the system by moving hand-piece 24, to terminate the application of ultrasound to a given area, or actuate manual controls 56 and 58 to apply ultrasound to an area after the system has determined that the calculated dosage has been delivered.

Turning now to FIG. 9, the physiology associated with the inventive application of ultrasound may be understood. A cross section of skin and subdermal tissue of a patient having cellulite is shown generally at 410. The tissue at 410 comprises the epidermis 411, the dermis 412 and subcutaneous adipose tissue 413. The interface 414 between the dermis 412 and subcutaneous adipose tissue 413 is a thin layer of connective tissue which loosely holds the fat cells comprising the adipose tissue 413 in juxtaposition to one another. The fibrous matrix may be viewed much as a fishnet having an elastic capability. Due to the collagen composition of the layer of connective tissue at the interface, the elastic matrix may be more or less irregularly deformable by fat cell aggregations 415 (fat lobules) comprising the underlying subcutaneous adipose tissue. In patients with cellulite, the fibrous layer comprising the interface 414 appears to be less substantial and more deformable than in people who do not present cellulite.

In order to strengthen the fibrous layer comprising the interface 414 to prevent the protrusion therethrough of the fat lobules 415 comprising adipose tissue 413, a means may be employed to damage tissue in the vicinity of the dermis-subcutaneous tissue interface. Upon disruption of cells in the layer comprising the interface 414, the protective bodily systems produce an infusion of cells which, in part, remove the debris and cause some degree of inflammation. The area is reinforced with an additional amount of connective tissue deposition as part of the tissue repair and healing phase. This phase is followed by a period of maturation of the newly deposited connective tissue, thereby resulting in contracture and tightening of the injured tissues and the tissue overlying dermis-epidermis interface. This newly deposited connective tissue matrix may be used to strengthen the natural fibrous layer between the dermis and subcutaneous tissue.

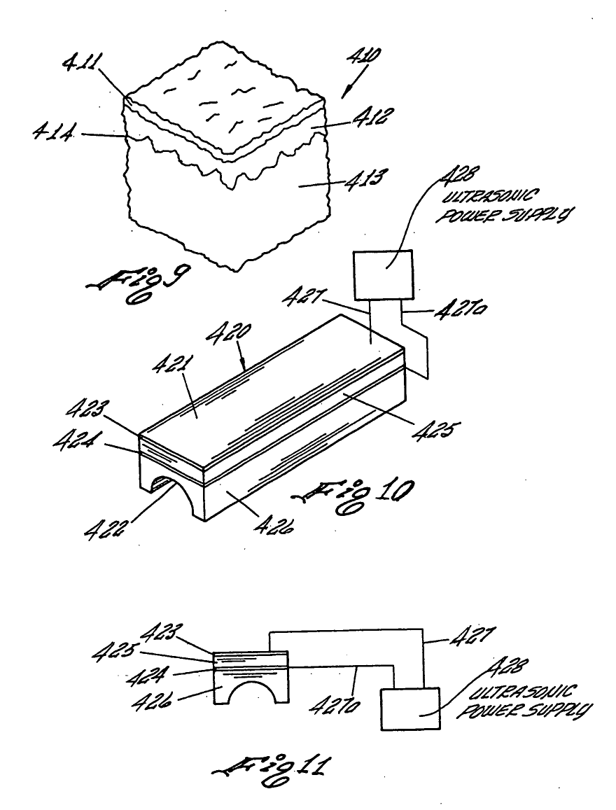

FIG. 10 shows the skin-contacting portion 420 of an ultrasonic vibrator hand-piece in perspective view which can focus ultrasonic energy at a layer of tissue beneath the epidermis and is useful in the embodiment of FIGS. 2-4. The skin-contacting portion 420 generally comprises an elongate member having an upper surface 421 and a concave lower surface 422. Conductive layers 423 and 424 provide electrically conductive means for applying an alternating electric field across a piezoelectric crystal 425 which is in mechanical vibratory communication with a metallic member 426. The electrode 420 receives an alternating voltage through leads 427 and 427a from an ultrasonic power source 428. The electrode 420 is symmetric along its length as shown in FIG. 11.

FIG. 11 is an end-on view of the ultrasonic probe of FIG. 10 showing the curvature of the concave surface. The curvature of the concave surface 422 of the skin-contacting portion of the ultrasonic hand-piece is adapted to focus ultrasonic energy applied to the upper skin surface to a line located a distance D below the upper surface of the skin. The distance D is preferably the thickness of the dermis with the region of cell disruption limited to a narrow vertical depth of about 0.5-5 mm but the capable of being focused to a depth extending an additional 2-3 cm below the dermis to achieve the desired result for the treatment of cellulite, depending upon the clinical situation. The ultrasonic energy may be focused within the dermis if such is required for the treatment of laxed or flaccid tissue (dermal-epidermal aytids).

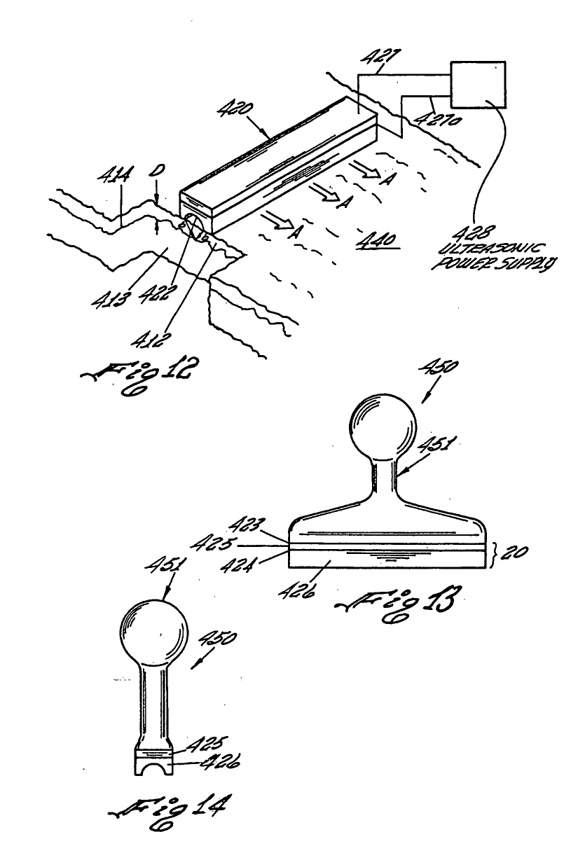

FIG. 12 is a schematic, partially cutaway diagram of the skin 440 showing the layer of tissue comprising the dermis 412, the layer of subcutaneous adipose tissue 413 and the interface 414 between the respective aforesaid layers of tissue. The concave surface 422 of the skin-contacting portion 420 of the ultrasonic vibrator hand-piece 450 (see FIGS. 13 and 14) is brought into contact with the surface of the skin 440. The ultrasonic power source 428 applies an alternating electrical voltage to conductive layers 423 and 424 thereby inducing the piezoelectric crystal 425 sandwiched therebetween to mechanically vibrate at an ultrasonic frequency, preferably in the 500 KHZ-1 MHZ range. The skin-contacting portion 420 is advanced across the skin in a direction A (shown by the broad arrows A) thereby covering the area of the skin presenting cellulite. The ultrasonic vibratory waves indicated at the thin arrows b are focused to have maximum amplitude within a band of tissue which band has a thickness which includes tissue at or near the interface 414 a distance D beneath the skin surface. In practice, the skin-contacting portion 420 of the ultrasonic vibratory hand-piece 450 is attached to a handle portion 451 to facilitate manipulation of the device as shown in FIGS. 13 and 14. The handle portion 451 is adapted to be affixed to the upper surface 421 of the skin-contacting portion 420 and grasped by a hand. FIG. 13 is a front elevational view of the hand-piece 450. A side elevational view of the hand-piece 450 is shown in FIG. 14. It may be advantageous to employ a means for mechanically displacing the protruding fat lobules downwards into the main portion of subcutaneous adipose tissue either prior to, during ultrasonic treatment or immediately following treatment. The latter may be accomplished by applying a compression dressing comprising a smooth anatomically conforming plate to the skin area overlying the treated area and applying pressure thereto with an elastic member compressed thereagainst. The method described hereinabove obviates the need for the intraoperative cooling of the skin during treatment of the target layer of tissue and does not rely on the temperature induced cross linking of extant collagen in order to provide a reinforced layer of tissue.

While an illustrative embodiment of the invention has been described, it is, of course, understood that various modifications will become apparent to those of ordinary skill in the art in view of the above description. Such modifications are within the spirit and scope of the invention which is limited and defined only by the appended claims.

1. Apparatus for treating cellulite under the surface of the skin of a patient, comprising:
a surgical ultrasonic energy source;
an ultrasonic energy transmitting surface, said ultrasonic energy transmitting surface being driven by said surgical ultrasonic energy source, said ultrasonic energy transmitting surface being configured to concentrate ultrasonic energy in the layer of connective tissue lying at the interface between the dermis and the subcutaneous adipose tissue, said surgical ultrasonic energy source being of sufficient power to cause disruption of said layer of connective tissue lying at the interface between the dermis and the subcutaneous adipose tissue during a dosage period;
an imaging ultrasonic energy source;
an imaging ultrasonic energy transmitting surface driven by said imaging ultrasonic energy source;
an imaging ultrasonic energy detector coupled to receive the reflected output of said imaging ultrasonic energy transmitting surface;
a computing device coupled to the output of said imaging ultrasonic energy detector;
a program resident in said computing device configuring said computing device to generate an image of tissues under the surface of the skin of a patient;
a display for displaying said image of tissues under the surface of the skin of said patient wherein said display provides the person implementing the treatment with first and second images, said first image being a before ultrasound treatment three dimensional or other image and said second image being an after ultrasound treatment image of the tissue being treated;
a applicator housing configured to be held by the hand of an individual, said imaging ultrasonic energy detector being mounted on said applicator housing, said imaging ultrasonic energy transmitting surface being mounted on said applicator housing, and said surgical ultrasonic energy transmitting surface being mounted on said applicator housing.

2. Apparatus for treating cellulite as in claim 1, wherein said concentration of electronic energy during said dosage period results in a dosage sufficient to result in the necrosis of cells in said connective tissue and cause the introduction of fibroblasts and the formation of new connective tissue sufficient to reinforce the layer of connective tissue to a degree where the strength of the connective tissue exceeds the strength of the connective tissue prior to the application of the dosage of ultrasound.

3. Apparatus for treating cellulite as in claim 1, wherein said surgical ultrasonic energy transmitting surface is driven by a piezoelectric device.

4. Apparatus for treating cellulite as in claim 1, wherein said ultrasonic energy transmitting surface is configured to be in contact with the surface of the skin of said patient.

5. Apparatus for treating cellulite as in claim 1, wherein said concentration of ultrasonic energy focuses that energy in a linear area.

6. Apparatus for treating cellulite as in claim 1, wherein said concentration of electronic energy during said dosage period results in a dosage sufficient to result in cell infusion and the formation of new connective tissue sufficient to reinforce the layer of connective tissue to a degree where the strength of the connective tissue exceeds the strength of the connective tissue prior to the application of the dosage of ultrasound.

7. Apparatus for treating cellulite as in claim 1, further comprising a pressure applying member configured to apply pressure to the skin area overlying the surface of the skin of the patient being treated.

8. Apparatus for treating cellulite as in claim 7, wherein said pressure applying member comprises a rigid member bearing against the skin of the patient and a pressure member urging said rigid member against the skin of the patient.

9. Apparatus for treating cellulite as in claim 1, wherein said ultrasonic energy is concentrated by being focused to have maximum amplitude within a band of tissue, said band of tissue having a thickness, said thickness including tissue at or near the interface between the adipose and dermal tissue layers.

10. Apparatus for treating cellulite as in claim 1, wherein said concentration of ultrasonic energy focuses that energy in selectable portions of a linear area.

11. Apparatus for treating cellulite as in claim 1, wherein said display is mounted on said applicator housing.

12. Apparatus for treating cellulite as in claim 1, wherein said imaging ultrasonic energy source and surgical ultrasonic energy source are alternately actuated.

13. Apparatus for treating cellulite as in claim 1, wherein said program determines and plans the administering of ultrasound and said applicator housing comprises indicators that guide directional movement of the applicator housing by the person implementing the treatment.

14. Apparatus for treating cellulite as in claim 1, wherein said program determines and plans the administering of ultrasound and said applicator housing comprises indicators that guide directional movement of the applicator housing by the person implementing the treatment, and further comprising an accelerometer coupled to said computing device to input information respecting applicator housing movement during the application of treatment ultrasound to said computing device.

15. Apparatus for treating cellulite as in claim 1, wherein said program determines and plans the administering of ultrasound and said applicator housing comprises indicators that guide directional movement of the applicator housing by the person implementing the treatment, and further comprising an accelerometer coupled to said computing device to input information respecting applicator housing movement during the application of treatment ultrasound to said computing device, and software associated with said computing device for generating a signal coupled to said display indicating the region of application of treatment ultrasound to tissue to be treated.

16. Apparatus for treating cellulite as in claim 1, wherein said program determines and plans the administering of ultrasound and said applicator housing comprises indicators that guide directional movement of the applicator housing by the person implementing the treatment, and actuators that overide the program-determined amplitude of treatment ultrasound in response to the actuation of said actuators by a person implementing the treatment.

17. Apparatus for treating cellulite under the surface of the skin of a patient, comprising an ultrasonic scanner comprising an surgical ultrasonic energy source and an ultrasonic energy transmitting surface, said ultrasonic energy transmitting surface being driven by said surgical ultrasonic energy source, said ultrasonic energy transmitting surface being configured, in cooperation with said ultrasonic scanner, to concentrate ultrasonic energy in the layer of connective tissue lying at the interface between the dermis and the subcutaneous adipose tissue, said surgical ultrasonic energy source being of sufficient power to cause disruption of said layer of connective tissue lying at the interface between the dermis and the subcutaneous adipose tissue during a dosage period, wherein said ultrasonic energy transmitting surface comprises a line of discrete energy transmitting members, and further comprising a logic device to control said surgical ultrasonic energy source to drive a selectable one of said discrete energy transmitting members in response to determined dosage amounts.

18. Apparatus as in claim 17, wherein the apparatus is caused to scan back and forth across the line of discrete energy transmitting members and successively alter cellular structure at the interface portion of the fat depot and the epidermis, whereby the person implementing the treatment may evaluate the progress of the treatment as it proceeds.

19. Apparatus as in claim 18, wherein the rate at which ultrasound is performed is commensurate with the rate at which the person implementing the treatment is able to monitor and evaluate the effects of ultrasound treatment.

20. Apparatus for treating cellulite as in claim 17, further comprising:
a computing device coupled to the output of said imaging ultrasonic energy detector;
a program resident in said computing device configuring said computing device to generate an image of tissues under the surface of the skin of a patient;
wherein said program determines and plans the administering of ultrasound and said applicator housing comprises indicators that guide directional movement of the applicator housing by the person implementing the treatment, whereby the apparatus indicates to the person implementing the treatment that the area has been treated and the person implementing the treatment is directed to move to the right or left.

21. Apparatus for treating cellulite under the surface of the skin of a patient, comprising an ultrasonic scanner comprising an surgical ultrasonic energy source and a surgical ultrasonic energy transmitting surface, said surgical ultrasonic energy transmitting surface being driven by said surgical ultrasonic energy source, said surgical ultrasonic energy transmitting surface being configured, in cooperation with said ultrasonic scanner, to concentrate ultrasonic energy in the layer of connective tissue lying at the interface between the dermis and the subcutaneous adipose tissue, said surgical ultrasonic energy source being of sufficient power to cause disruption of said layer of connective tissue lying at the interface between the dermis and the subcutaneous adipose tissue during a dosage period, and further comprising an imaging ultrasonic energy source, an imaging ultrasonic energy transmitting surface driven by said imaging ultrasonic energy source, an imaging ultrasonic energy detector coupled to receive the reflected output of said imaging ultrasonic energy transmitting surface, a computing device coupled to the output of said imaging ultrasonic energy detector, a program resident in said computing device configuring said computing device to generate an image of tissues under the surface of the skin of a patient, and a display on said scanner for displaying said image of tissues under the surface of the skin of said patient.

22. Apparatus as in claim 21, wherein said display provides the person implementing the treatment with first and second images, said first image being a before ultrasound treatment three dimensional or other image and said second image being an after ultrasound treatment image of the tissue being treated.

23. Apparatus as in claim 22, further comprising a scanner, wherein said display is mounted on said scanner, said imaging ultrasonic energy detector is mounted on said scanner, said imaging ultrasonic energy transmitting surface is mounted on said scanner, and said surgical ultrasonic energy transmitting surface is mounted on said scanner.
